(19) United States
(12) Patent Application Publication
(10) Pub. No.: US 2021/0292851 A1
(43) Pub. Date: Sep. 23, 2021

(54) METHOD OF MONITORING EFFECTIVENESS OF IMMUNOTHERAPY OF CANCER PATIENTS

(71) Applicants: Roche Sequencing Solutions, Inc., Pleasanton, CA (US); Genentech, Inc., South San Francisco, CA (US)

(72) Inventors: Aarthi Balasubramanyam, Sunnyvale, CA (US); Yuqiu Jiang, San Ramon, CA (US); John Palma, Alamo, CA (US); Namrata Patil, South San Francisco, CA (US); Johnny Wu, Castro Valley, CA (US); Stephanie Yaung, San Jose, CA (US); Wei Zou, San Carlos, CA (US)

(21) Appl. No.: 17/263,708

(22) PCT Filed: Jul. 29, 2019
(86) PCT No.: PCT/EP2019/070308

§ 371 Date: Jan. 27, 2021

Related U.S. Application Data
(60) Provisional application No. 62/711,413, filed on Jul. 27, 2018.
(60) Provisional application No. 62/844,478, filed on May 7, 2019.

(51) Int. Cl. C12Q 1/6886; C12Q 1/6869

(52) U.S. Cl. CPC C12Q 1/6886; C12Q 2600/156; C12Q 2600/106; C12Q 1/6869

(57) ABSTRACT

The invention is a method of determining a likelihood that a cancer patient will respond to immunotherapy based on a mutation metric obtained by sequencing a small panel of nucleic acid targets in patient's cell-free DNA.

### FIELD OF THE INVENTION

The invention relates to the field of oncology. More specifically, the invention relates to the field of nucleic acid-based testing of cancer patients receiving or selected as candidates for receiving cancer immunotherapy.

### BACKGROUND OF THE INVENTION

Immunotherapy is a promising novel field of oncology. Immune checkpoint inhibition such as anti-PD1 or anti-PD L1 antibody treatments have been shown to be effective in patients with lung and other types of cancers. However, the survival benefit of immunotherapy varies from patient to patient. There is an unmet need for predictive biomarkers to guide therapy decisions in patients who are potential candidates for immunotherapy.

### SUMMARY OF THE INVENTION

In some embodiments, the invention is a method of treatment of a cancer patient comprising the steps of: isolating nucleic acids from a cell-free blood sample obtained from the patient; in the isolated nucleic acid, determining the sequence of at least a portion of each of the biomarkers listed in Table 1; comparing the sequence to the reference sequence and identifying mutations; determining a mutation metric from the identified mutations, administering an immunotherapy agent if the mutation metric is high and not administering the immunotherapy agent if the mutation metric is low. The sequence of at least a portion of each of the biomarkers listed in Table 1 is determined by a method comprising: attaching adaptors comprising barcodes to the isolated nucleic acid to generate adapted nucleic acid; amplifying the adapted nucleic acid to generate amplified non-uniquely tagged progeny polynucleotides; contacting the amplified nucleic acid with capture probes to capture the amplified nucleic acid comprising at least a portion of each of the biomarkers listed in Table 1; sequencing the captured nucleic acid. The mutation metric may be selected from mutation burden, allele frequency (AF), maximum allele frequency (MaxAF), number of mutant molecules per milliliter (MMPM) and maximum MMPM (MaxMMPM). Mutation burden can be determined as a ratio of the number of mutations identified to the number of bases of nucleic acid sequenced. Maximum allele frequency (MaxAF) can be determined as the highest allele frequency of a single mutation among all mutations detected within a single sample. The number of mutant molecules per milliliter (MMPM) can be determined as MMPM=AF×HG/V where AF is allele frequency of a particular allele; HG is input haploid human genome equivalent calculated as (extracted mass of DNA in nanograms)×(300 human genome equivalents/nanograrn); and V is the volume of plasma in milliliters; and maximum number of mutant molecules per milliliter (MaxMMPM) can be determined as MaxMMPM=maxAF×HG/V where maxAF is maximum allele frequency among the alleles in the sample; HG is input haploid human genome equivalent calculated as (extracted mass of DNA in anograms)×(300 human genome equivalents/nanogram); and V is the volume of plasma in milliliters.

In some embodiments, the mutation metric is high if it falls on or above the median of the mutation metric of patients with the same cancer type. In some embodiments, the mutation metric is high if it falls on or above the top quartile of the mutation metric of patients with the same cancer type.

In some embodiments, the patient is diagnosed with one of carcinoma, sarcoma, myeloma, leukemia or lymphoma. In some embodiments, the immunotherapy agent is an immunomodulating antibody, an immune checkpoint inhibitor antibody, an antibody is selected from an anti-PD-1, anti-PD-L1 and anti-CTLA-4 antibody, including nivolumab administered every 4 weeks at 480 mg or every 2 weeks at 240 mg; ipilimumab administered at 10 mg/kg every 3 weeks, or atezolizumab administered at 1200 mg every 3 weeks.

In some embodiments, the invention is a method of determining whether a cancer patient is likely to have a benefit from a therapy with an immune checkpoint inhibitor, the method comprising the steps of: isolating nucleic acids from a cell-free blood sample obtained from the patient; in the isolated nucleic acid, determining the sequence of at least a portion of each of the biomarkers listed in Table 1; comparing the sequence to the reference sequence and identifying mutations; determining a mutation metric from the identified mutations; determining that the patient is likely to have a benefit from a therapy with an immune checkpoint inhibitor if the mutation metric is high and determining that the patient is not likely to have a positive response to a therapy with an immune checkpoint inhibitor if the mutation metric is low. The sequence of at least a portion of each of the biomarkers listed in Table 1 may be determined by a method comprising: attaching adaptors comprising barcodes to the isolated nucleic: acid to generate adapted nucleic acid; amplifying the adapted nucleic acid to generate amplified non-uniquely tagged progeny polynucleotides; contacting the amplified nucleic acid with capture probes specific for least a portion of each of the biomarkers listed in Table 1 to capture the amplified nucleic acid; sequencing the captured nucleic acid. The mutation metric may be mutation burden, allele frequency (AF), maximum allele frequency (MaxAF), number of mutant molecules per milliliter (MMPM) and maximum MMPM (MaxMMPM). In some embodiments, the mutation metric is high if it falls above the median of the mutation metric of patients with the same tumor type and the mutation metric is low if it falls below the median of the mutation metric of patients with the same tumor type. In some embodiments, the cell-free blood sample is obtained from the patient during the therapy with an immune checkpoint inhibitor, e.g., at midpoint during then therapy.

In some embodiments, the method further comprises a step of administering the checkpoint inhibitor to the patient if it has been determined that the patient will benefit from the therapy with the inhibitor. In some embodiments, the method further comprises a step of ceasing administration of the checkpoint inhibitor to the patient if it has been determined that the patient will not benefit from the therapy with the inhibitor.

In some embodiments, the method further comprises a step of administering an alternative therapy (e.g., chemotherapy with a cytotoxic compound) to the checkpoint inhibitor to the patient if it has been determined that the patient will not benefit from the therapy with the inhibitor.

### BRIEF DESCRIPTION OF THE DRAWINGS

FIG. 1 shows the relationship between mutant allele frequency and progression-free survival (PFS) using a 50% (median) cut-off.

FIG. 2 shows the relationship between mutant allele frequency and progression-free survival (PFS) using a 75% (upper quartile) cut-off.

FIG. 3 shows the relationship between maximum allele frequency (MaxAF) and progression-free survival (PFS) using a 50% (median) cut-off.

FIG. 4 shows the relationship between the metric of maximum mutant molecule per MMPM) and overall survival (OS) using a 50% (median) cut-off.

### DETAILED DESCRIPTION OF THE INVENITON

Definitions

The following definitions are not limiting but merely aid in understanding this disclosure.

The term “biomarker” is used herein to describe a nucleotide sequence that contains information relevant to the biological or clinical phenomenon. For example, the information may be a mutation status of the sequence. The biomarker can be a gene (including coding sequence, regulatory sequence, intron or a splice site) or an intergenic region. The clinical phenomenon can be the presence of malignant cells, e.g., tumor cells in a patient's sample.

The term “circulating tumor DNA (ctDNA)” is used herein to describe a portion of cell-free DNA found in human blood plasma or serum that originates from the tumor. Circulating tumor DNA is distinguished from non-tumor DNA by the mutations characteristic of the tumor. In the context of the present invention, detecting ctDNA means detecting mutated cell-free DNA.

The term “cancer driver gene” is used herein to describe one whose mutations and resulting aberrant activity increase net cell growth. Some examples of cancer driver genes include EGFR, KRAS, BRAF, ALK.

The term “cancer immunotherapy” refers to therapy aimed at using the patient's immune system to target tumor cell. Cancer immunotherapy includes administration of immune checkpoint inhibitors. Current non-limiting examples of checkpoint inhibitors include PD-1 inhibitors (pembrolizumab (Keytryda®), nivolumab (Opdivo®) and cemiplimab (Libtayo®)), PD-L1 inhibitors (atezolizumab (Tecentriq®), avelumab (Bavencio®) and durvalumab (Infinzi®)) and CTLA-4 inhibitors (ipilimumab (Yervoy®)).

The term “mutation metric” refers to a method of assessing the number of mutations in the patient's sample. The term “allele frequency (AF)” refers to a mutation metric reflecting the frequency of a mutant allele among the normal allele sequences. The term “maximum allele frequency (maxAF)” refers to a mutation metric calculated as the highest AF among all the different alleles (mutant sequences) found in the sample. The term “mutant molecules per milliliter (MMPM)” refers to a mutation metric reflecting the number of mutant alleles in a volume of plasma. The term “maximum MMPM (maxMMPM)” refers to a mutation metric calculated as the highest MMPM among all the different alleles (mutant sequences) found in the sample.

The term “OS” refers to the time of Overall Survival for a patient.

The term “PFS” refers to the time of Progression Free Survival for a patient.

The term “whole genome sequencing of WGS” is used herein to describe sequencing of the entire genome of a cell or organism from which a cells or cells are derived. The term “whole exome sequencing of WES” is used herein to describe sequencing of all the exons present in all the genes of the genome. Both WGS and WES on human or other higher order vertebrate genomes are performed using massively parallel sequencing methods (next generation sequencing methods) capable of gathering and storing large amounts of sequence information.

Tumor cell are known to accumulate somatic mutations during tumor development and progression. Tumor Mutation Burden (TMB) is defined as a number of mutations in a tumor sample from a patient. TMB is an established biomarker for chemotherapies and cancer immunotherapies for an array of solid metastatic malignancies. TMB is often measured by whole exome sequencing (WES) or by sequencing of multi-megabase panels. In recent studies, a panel comprising 1.2 megabase of the genome was analyzed for mutations. Hybridization capture of exonic regions from 185, 236, 315, or 405 cancer-related genes and select introns from 19, 28, or 31 genes commonly rearranged in cancer was applied to ≥50 ng of DNA extracted from formalin-fixed, paraffin-embedded clinical cancer specimens. Chalmers et al. (2017) Analysis of 100,000 human cancer genomes reveals the landscape of tumor mutational burden, Genome Medicine (2017) 9:34, Goodman et al., (2017) Tumor Mutational Burden as an Independent Predictor of Response to Immunotherapy in Diverse Cancers, doi: 10.1158/1535-7163.MCT-17-0386, Maia, M. C., et al.,(2018) Relationship of tumor mutational burden (TMB) to immunotherapy response in metastatic renal cell carcinoma (mRCC), Journal of Clinical Oncology 36, no. 6_suppl (Feb. 20, 2018) 662-662.

Recently, a new more practical, non-invasive method of assessing TMB has been developed and described in an International Application Ser. No. PCT/EP2019/061328 filed on May 3, 2019 titled “Surrogate Marker and Method for Tumor Mutation Burden Measurement”. The new method utilizes a targeted sequencing panel to measure mutations found in circulating tumor DNA (ctDNA) of cancer patients. The method employs greater depth of sequencing compared to e.g., tissue-based sequencing and utilizes a smaller targeted sequencing panel that enriches the commonly mutated genes in solid tumors. In one embodiment, the mutation count is assessed using the AVENIO ctDNA Surveillance Kit (Roche Sequencing Solutions, Inc., Pleasanton, Calif.), a targeted next-generation sequencing panel of 198 kilobases. The panel was designed to maximize the number of relevant mutations to be detected in lung and colorectal cancers interrogating only 198 kb of the human genome. Although a smaller length of the genome is sequenced and the samples include only cell-free DNA, the methods has been shown to adequately assess the status of tumor DNA.

The present invention is a method to determine likelihood that a patient will respond to immunotherapy.

Effectiveness of a therapy can be expressed using several parameters including prolongation any of the following time intervals: Overall survival (OS), Progression Free Survival (PFS), Recurrence Free Survival (RFS), and Total Time to Recurrence (TTR). Determining that a therapy is likely to be effective can be expressed as a likelihood that the above mentioned time periods will be prolonged or extended. The present invention discloses an early surrogate of these indicators of effectiveness. The novel method disclosed herein provides an early clinical efficacy signal and enables treatment decisions. In some embodiments, the invention is a method of determining whether Overall survival (OS) will be extended following administration of immune checkpoint inhibitors.

The invention utilizes a blood sample from a patient. The patient may be a cancer patient or a suspected cancer patient wherein the cancer is selected from carcinoma (including bladder, lung, renal carcinoma or melanoma), sarcoma, myeloma, leukemia or lymphoma. In some embodiments, the patient is a non-small cell lung cancer (NSCLC) patient. The sample can include any fraction of blood, e.g., serum or plasma, which contains cell-free DNA including circulating tumor DNA (cfDNA or ctDNA). The sample is taken prior to invitation of immunotherapy. In some embodiments, the sample is taken serially at various times during treatment, e.g., before and after surgery or before, after and during a chemotherapy regimen, a chemoradiation therapy regimen or a regimen of adjuvant therapy. In some embodiments, the sample is taken serially during therapy to assess effectiveness of the therapy. The blood sample can be collected by a suitable means that preserves the cell-free DNA therein, including collecting blood or blood plasma in a preservative medium.

In some embodiments, the invention comprises a step of DNA isolation. Cell-free DNA (cfDNA) may be extracted from patients' liquid biopsy samples (including blood or plasma samples) using solution-based or solid-phase based nucleic acid extraction techniques. Nucleic acid extraction can include detergent-based cell lysis, denaturation of nucleoproteins, and optionally removal of contaminants. Solution based nucleic acid extraction methods may comprise salting out methods or organic solvent or chaotrope methods. Solid-phase nucleic extraction methods can include but are not limited to silica resin methods, anion exchange methods or magnetic glass particles and paramagnetic beads (KAPA Pure Beads, Roche Sequencing Solutions, Pleasanton, Calif.) or AMPure beads (Beckman Coulter, Brea, Calif.) A cfDNA extraction method may utilize a step of applying preservative to a blood or plasma sample to prevent lysis of residual blood cells present in the sample, for example preservatives described in U.S. Pat. No. 5,849,517 or U.S. Pat. No. 8,586,306 can be used including imidazolidinyl urea, diazolidinyl urea, EDTA or combinations thereof. Cell-free DNA present in solution can be bound to a solid support (beads or particles) present in solution or packed in a column, or membrane where the DNA may undergo one or more washing steps to remove contaminants including proteins, lipids and fragments thereof from the sample. Finally, the bound cfDNA can be released from the solid support, column or membrane and stored in an appropriate buffer until ready for further processing.

In some embodiments, the invention comprises a step of ligating an adaptor to the isolated cfDNA. The adaptor may be ligated to the ends of a double stranded DNA molecule. Prior to ligation, the cfDNA molecule may be enzymatically treated to create blunt ends (end repair) and further treated to create an overhang to facilitate adaptor ligation (A-tailing). Commercially available kits for performing adaptor ligation include AVENIO ctDNA Library Prep Kit or KAPA HyperPrep and HyperPlus kits (Roche Sequencing Solutions, Pleasanton, Calif.). In some embodiments, the adaptor-ligated (adapted) DNA may be separated from excess adaptors and unligated DNA

In some embodiments, the adaptor comprises one or more barcodes. A barcode can be a multiplex sample ID (MID) used to identify the source of the sample where samples are mixed (multiplexed). The barcode may also serve as a unique molecular ID (UID) used to identify each original molecule and its progeny. The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the invention, the barcodes are between about 4-20 bases long so that between 96 and 384 different adaptors, each with a different pair of identical barcodes are added to a human genomic sample. A person of ordinary skill would recognize that the number of barcodes depends on the complexity of the sample (i.e., expected number of unique target molecules) and would be able to create a suitable number of barcodes for each experiment.

Detecting individual molecules typically requires molecular barcodes such as described in U.S. Pat. Nos. 7,393,665, 8,168,385, 8,481,292, 8,685,678, and 8,722,368. A unique molecular barcode is a short artificial sequence added to each molecule in the patient's sample typically during the earliest steps of in vitro manipulations. The barcode marks the molecule and its progeny. The unique molecular barcode (UID) has multiple uses. Barcodes allow tracking each individual nucleic acid molecule in the sample to assess, e.g., the presence and amount of circulating tumor DNA (ctDNA) molecules in a patient's blood in order to detect and monitor cancer without a biopsy (Newman, A., et al., (2014) An ultrasensitive method for quantitating circulating tumor DNA with broad patient coverage, Nature Medicine doi:10.1038/nm.3519).

Unique molecular barcodes can also be used for sequencing error correction. The entire progeny of a single target molecule is marked with the same barcode and forms a barcoded family. A variation in the sequence not shared by all members of the barcoded family is discarded as an artifact and not a true mutation. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments, the invention comprises an amplification step. The double-stranded DNA fragments prepared by the method described herein or optionally adapted nucleic acids can be amplified prior to sequencing This step can involve linear or exponential amplification, e.g., PCR. Amplification may be isothermal or involve thermocycling. In some embodiments, the amplification is exponential and involves PCR. In some embodiments, universal primers are used, i.e., a pair of primers that hybridizes to a universal primer binding site in the adaptor present on all target sequences in the sample. All molecules in the library having the same adaptor containing a universal primer binding site can be amplified with the same set of primers. The number of amplification cycles where universal primers are used can be low but also can be 10, 20 or as high as about 30 or more cycles, depending on the amount of product needed for the subsequent steps. Because PCR with universal primers has reduced sequence bias, the number of amplification cycles need not be limited to avoid amplification bias.

In some embodiments, the method comprises only one round of amplifying adapted nucleic acids prior to sequencing. In other embodiments, the method comprises additional rounds of amplification, e.g., after enrichment or capture as described herein.

In some embodiments, the invention further comprises a step of target enrichment. In some embodiments, the method utilizes a pool of oligonucleotide probes (e.g., capture probes) to capture a panel of biomarkers. The invention utilizes a biomarker panel, including a gene panel or a mutation panel or a somatic variant panel. The mutations may include single-nucleotide variations (SNVs), deletions and insertions (in-dels) that correspond to on-sense missense and frame-shift mutations if they occur in the coding regions of genes. Other types of mutations include gene fusions and translocations. The selection, size and content of such panels has been described e.g., in International App. No. PCT/US2015/049838 titled “Identification and Use of Circulating Tumor Markers.” In some embodiments, the invention includes determining the sequence of the biomarkers in the panel, e.g., the genes listed in Table 1. In some embodiments, the entire sequence of a gene is determined. In other embodiments, the entire coding sequence of a gene is determined. In other embodiments, only the sequence of a portion of the gene known to undergo mutagenesis in cancer is determined. In yet other embodiments, the biomarker is not associated with a coding sequence but is associated with a regulatory sequence or a sequence of unknown function known to be mutated in human tumors.

In some embodiments, the invention utilizes a biomarker panel, such as AVENIO® ctDNA Analysis Kit (Roche Sequencing Solutions, Inc., Pleasanton, Calif.) that is capable of analyzing the blood of patients after surgery to identify whether patients have circulating tumor DNA (ctDNA). In some embodiments, a panel that represents NCCN guideline recommended biomarkers for targeted therapies (17 genes) is used. In other embodiments, a broader panel further including therapy resistance markers (total 60 genes) is used. In yet another example, a broader panel further including cancer hotspot mutations (total 180 genes) is used (see the patent applications “Identification and Use of Circulating Tumor Markers”, supra). The composition of the biomarker panel in AVENIO® ctDNA Analysis Kit is shown in Table 1.

TABLE 1
Composition of the biomarker panel

| | | | | | | | |
|---|---|---|---|---|---|---|---|
| ABCC5 | CSMD1 | FAT1 | HTR1E | MAP7D3 | PIK3CA | SV2A | AR |
| ABCG2 | CSMD3 | FBN2 | HTR2C | MKRN3 | PIK3CG | T | CCND1 |
| ACTN2 | CTNNB1 | FBXL7 | IFI16 | MMP16 | PKHD1L1 | THSD7A | CCND2 |
| ADAMTS12 | CTNND2 | FBXW7 | IL7R | MTX1 | POLE | TIAM1 | CCND3 |
| ADAMTS16 | CYBB | FCRL5 | INSL3 | MYH7 | POM121L12 | TMEM200A | CD274 |
| ARFGEF1 | DCAF12L1 | FOXG1 | ITGA10 | MYT1L | PREX1 | TNFRSF21 | CDK4 |
| ASTN1 | DCAF12L2 | FRYL | ITSN1 | NAV3 | PTPLA | TNN | CDKN2A |
| ASTN2 | DCAF4L2 | GBA3 | KCNA5 | NEUROD4 | RALYL | TNR | ESR1 |
| AVPR1A | DCLK1 | GBP7 | KCNB2 | NFE2L2 | RFX5 | TRHDE | FBXW7 |
| BCHE | DCSTAMP | GJA8 | KCNC2 | NLGN4X | RIN3 | TRIM58 | KEAP1 |
| BPIFB4 | DDI1 | GPR139 | KCNJ3 | NLRP3 | RNASE3 | TRPS1 | MLH1 |
| C6 | DLGAP2 | GRIA2 | KCTD8 | NMUR1 | ROBO2 | UGT3A2 | MSH2 |
| C6orf118 | DMD | GRIK3 | KEAP1 | NOL4 | SEMA5B | USH2A | MSH6 |
| CA10 | DNTTIP1 | GRIN2B | KIAA1211 | NPAP1 | SLC18A3 | USP29 | NF2 |
| CACNA1E | DOCK3 | GRIN3B | KIF17 | NR0B1 | SLC39A12 | VPS13B | PDCD1LG2 |
| CDH12 | DSC3 | GRM1 | KIF19 | NRXN1 | SLC6A5 | WBSCR17 | PMS2 |
| CDH18 | DSCAM | GRM5 | KLHL31 | NXPH4 | SLC8A1 | WIPF1 | PTEN |
| CDH8 | EGFLAM | GRM8 | KPRP | NYAP2 | SLITRK1 | WSCD2 | RB1 |
| CDH9 | EPHA5 | GSX1 | LPPR4 | OPRD1 | SLITRK4 | ZC3H12A | SMAD4 |
| CDKN2A | EPHA6 | HCN1 | LRFN5 | P2RY10 | SLITRK5 | ZFPM2 | SMO |
| CHRM2 | EYS | HCRTR2 | LRP1B | PAX6 | SLPI | ZIC1 | STK11 |
| CNTN5 | FAM135B | HEBP1 | LRRC7 | PCDH15 | SMAD4 | ZIC4 | VHL |
| CNTNAP2 | FAM151A | HECW1 | LRRTM1 | PDYN | SOX9 | ZNF521 | APC |
| CPXCR1 | FAM5B | HS3ST4 | LRRTM4 | PDZRN3 | SPTA1 | ZSCAN1 | BRCA1 |
| CPZ | FAM5C | HS3ST5 | LTBP4 | PGK2 | ST6GALNAC3 | N/KRAS | BRCA2 |
| CRMP1 | FAM71B | HTR1A | MAP2 | PHACTR1 | STK11 | MET | EGFR |
| ALK | PDGFRA | RAF1 | JAK3 | NFE2L2 | TSC2 | MTOR | PIK3R1 |
| BRAF | RET | RNF43 | KDR | NTRK1 | TSC1 | MAP2K2 | PIK3CA |
| DPYD | ROS1 | TERT | MAP2K1 | PDGFRB | KIT | UGT1A1 | PTCH1 |
| | | promoter | | | | | |

In some embodiments, the invention further includes a step of improving the biomarker panel based on the results obtained from the clinical samples of patients undergoing immunotherapy such as therapy with an immune checkpoint inhibitor. In some embodiments, the invention includes the steps of analyzing the correlation between the presence of a biomarker in the cell-free DNA from a statistically significant number of patients and A) RFS, B) TTR (or DFS), and C) OS experienced after receiving immunotherapy. The biomarkers showing a predictive correlation are to be included in the panel. The biomarkers not showing a statistically significant predictive correlation are to be excluded from the panel.

In the context of the present invention, the sequence of a biomarker can be determined via any suitable method known in the art. The suitable method would have sufficient accuracy, e.g., sensitivity and specificity to detect rare sequences with a low rate of errors. In some embodiments, the sequencing method includes an error correction step, such as use of molecular barcodes, error stereotyping and other chemical or computation methods of error suppression as described e.g., in see the patent applications “Identification and Use of Circulating Tumor Markers”, supra. The sequencing method may include a massively parallel sequencing method, including an array based sequencing (Illumina, San Diego, Calif.), an emulsion-based sequencing (ThermoFisher, Waltham, Mass.) an optical measurement based sequencing (Pacific BioSciences, Menlo Park, Calif.) or a nanopore-based sequencing (Roche Sequencing Solutions, Santa Clara, Calif.), or Oxford Nanopore (Oxford, UK).

The sequence data is compared to a reference genome sequence to determine mutations. In some embodiments, the reference sequence is the canonical human genome assembly e.g., I-IG38. Changes in the nucleic add sequence compared to the reference genome are added to determine a mutation metric. The mutation metric may be selected from mutation burden, total number of mutations, allele frequency (AF), maximum allele frequency (MaxAF), mutant molecules per milliliter (MMPM) or maximum MMPM (maxMMPM). The mutation burden is determined as a ratio of the number of mutations identified and the number of bases of nucleic acid sequenced, and the maximum allele frequency (MaxAF) is determined as the highest allele frequency of a single mutation among all mutations detected within a single sample. In some embodiments, one or more filters are applied to select mutations to be scored, i.e., added to the mutation score. In some embodiments, only mutations in the coding regions are scored. In some embodiments, only non-synonymous mutations in the coding regions are scored. In other embodiments, both non-synonymous and synonymous mutations in the coding regions are scored. In yet other embodiments, to enable detection of lower frequency mutation events, mutations in the cancer driver genes are excluded from scoring.

In some embodiments, the mutation metric is Mutant Molecules Per Milliliter (MMPM) as described e.g., in the international application PCT/EP2019/053250 filed on Feb. 11, 2019 for Using DNA Molecule Counts in Blood for Early Treatment Response Prediction. In some embodiments, MMPM is calculated according to Formula 1:

MMPM=AF×HG/V Formula 1

where
AF is allele frequency of a particular allele;
HG is input haploid human genome equivalent calculated as (extracted mass of DNA in nanograms)×(300 human genome equivalents/nanogram);
V is the volume of plasma in milliliters Max MMPM is the highest (maximum) MMPM among all the variants in the sample. MaxMMPM can be calculated according to Formula 2:

MaxMMPM=maxAF×HG/V Formula 2

In some embodiments, the mutation metric determined according to the instant invention is assessed as high or low. In some embodiments, the mutation metric is assessed al the population level wherein the relevant population consists of cancer patients diagnosed with the same type of cancer. In some embodiments, the population comprises patients diagnosed with NSCLC. In some embodiments, the population comprises patients undergoing immunotherapy. In some embodiments, the population comprises healthy individuals (controls). In some embodiments, the mutation metric is defined as low if it falls under a quantile in the population and the mutation metric is defined as high if it falls at or above the quantile in the population. The quantile may be a quartile, a tertile or a median. In some embodiments, the quantile is the upper quartile and the mutation metric is defined. as high if it falls above the third quartile in the population and the mutation metric is defined as low if it falls at or below the third quartile in the population. In some embodiments, the quantile is the median and the mutation metric is defined as high if it falls above the median in the population and the mutation metric is defined as low if it falls at or below the median in the population.

In some embodiments, the mutation metric is measured as a single value as described above while in other embodiments, a trend or change in the mutation metric over time is determined. In some embodiments, the patient samples are collected at different times during treatment and the values of the metric (e.g., MaxAF or MMPM are compared to determine increase or decrease in value.

In some embodiments, the invention also includes a step of assessing the status of a cancer in a patient. In some embodiments, the assessing includes identifying the patient as likely or not likely to experience a benefit from immunotherapy. The assessing is based on the mutation metric determined as described herein. In some embodiments, the invention includes a step of statistical analysis to determine whether a patient is likely to experience a benefit from immunotherapy. The benefit is selected from prolonging Progression Free Survival (PFS), Recurrence Free Survival (RFS), Total Time to Recurrence (TTR) and Overall Survival (OS).

In some embodiments, the invention also includes a step of recommending or administering therapy to a cancer in a patient. In some embodiments, the invention includes a step of determining whether the patient is likely to respond to therapy. In some embodiments, the invention comprises assessing the patient as described herein at each step of administering a therapy. In such embodiments, the method may comprise a step of recommending to continue or continuing the therapy or recommending to discontinue or discontinuing the therapy.

In some embodiments, the therapy is immunotherapy including immune checkpoint modulating therapy with immune checkpoint inhibitors (e.g., antibodies against PD-1, PD-L1, CTLA-4, and LAG-3). It has previously been reported that the overall number and rate of mutation and therefore of potential neoepitopes in cancer cells can be predictive of clinical response to immunotherapy, and particularly to immune checkpoint modulator therapy. (WO2016081947). The mutation estimate according to the present invention allows assessing tumor responsiveness to therapy particularly to immunotherapy such as immune checkpoint modulator therapy. In some embodiments, such therapy involves blockade of programmed cell death 1 (PD-1) using a composition comprising an anti-PD-1 antibody. In some particular embodiments, such therapy involves administration of one or more of nivolumab (BMS-936558), pembrolizumab (MK-3475). In some embodiments, the therapy involves administration of an antibody against the PD-1 ligand PD-L1 including atezolizumab (MPDL3280A), avelumab (MSB0010718C) or durvalumab (MEDI4736). In some embodiments, the therapy involves administration of an antibody against CTLA-4 including ipilimimab and tremelimumab. It has been demonstrated that for certain cancers, including small cell or non-small-cell carcinoma of the lung, bladder cancer, renal carcinoma, head and neck cancers, and melanoma patients with high numbers of mutations are more likely to benefit from treatment with immune checkpoint modulators than those patients with lower mutation loads. In some embodiments, patients with higher numbers of somatic mutations respond better to PD-1 blockade (e.g., with anti-PD-1 or anti-PD-L1 antibodies) than those patients with significantly lower overall mutations. (see WO2016081947). Somatic mutations such as the ones detected by the method disclosed herein comprise DNA alterations in somatic cells including cancer cells. In the present invention, sequence variations also detected in normal somatic cells (e.g., PBMC or peripheral blood mononuclear cells) are excluded from mutation scoring. These mutations are detected in the DNA released by the tumor cells into the blood stream (circulating tumor DNA or ctDNA). Somatic mutations in protein-coding regions can result in new epitopes recognized by the immune system (neoantigens). Upon lifting of the inhibition by PD-1 (e.g., by PD-1 or PD-L1 blocking agents listed above), the tumor comprising neoantigens is subject to attack by the patient's immune system.

In some embodiments, the invention provides methods for identifying cancer patients that are likely to respond favorably to treatment with an immune checkpoint modulator and treating the patient with an immune checkpoint modulator. The invention also provides methods for identifying cancer patients that are not likely to respond favorably to treatment with an immune checkpoint modulator and recommending that the patients not be treated with an immune checkpoint modulator or recommending that the patients not be treated with an alternative, such as a chemotherapy or chemoradiation therapy. Similarly, the invention provides methods for assessing the patient as described herein at more than one step including at each step of administering a therapy and based on the assessment, recommending to continue the therapy or recommending to discontinue the therapy and optionally, to start alternative therapy. Specifically, the invention is a method of treatment of a cancer patient comprising the steps of isolating nucleic acids from a cell-free blood sample obtained from the patient, in the isolated nucleic acid, determining the sequence of at least a portion of each of the biomarkers listed in Table 1, comparing the sequence to the reference sequence and identifying mutations, and further determining one or more of the mutation metrics selected from mutation burden, total number of mutations, the amount of mutations per volume of plasma (e.g., Mutant Molecules Per Milliliter, MMPM), maximum allele frequency (MaxAF) and maximum MMPM (maxMMPM) and then administering an immunotherapy agent if the mutation metric is high and not administering the immunotherapy agent if the mutation metric is low. The mutation burden is determined as a ratio of the number of mutations identified and the number of bases of nucleic acid sequenced. The maximum allele frequency (MaxAF) is determined as the highest allele frequency of a single mutation among all mutations detected within a single sample, the MMPM value is determined e.g., as described in the international application PCT/EP2019/053250 filed on Feb. 11, 2019 for Using DNA Molecule Counts in Blood for Early Treatment Response Prediction. In some embodiments, the immunotherapy agent is an anti-PD-L1 antibody such as atezolizumab administered every 3 weeks at 1200 mg, an anti-PD-1 antibody such as nivolumab administered every 4 weeks at 480 mg or every 2 weeks at 240 mg. In other embodiments, the immunotherapy agent is an anti-CTLA4 antibody such as ipilimumab administered at 10 mg/kg every 3 weeks.

In some embodiments, the method of treatment of a cancer patient comprises the steps described above except the step of isolating nucleic acids occurs at two or more time points in the course of administering the immunotherapy. The mutations in the samples collected at several points during the therapy are detected and scored in the cell-free fraction of the patient's blood as described herein. One or more mutation metrics is determined as described above followed by continuing to administer the immunotherapy agent if the mutation metric is high and discontinuing the administration of the immunotherapy agent if the mutation metric is low.

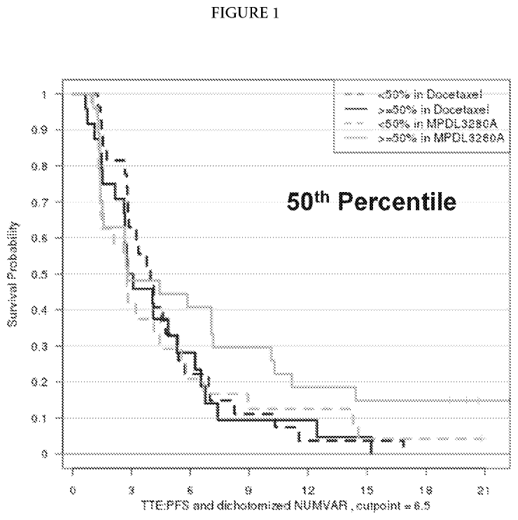

In one embodiment, baseline plasma samples can be collected from patients prior to treatment with immune checkpoint inhibitors, or one or more times during the treatment, including at a specific time point during the treatment e.g., after 6 weeks. Circulating tumor DNA (ctDNA) isolated from the patient samples can be analyzed to detect mutations in a select panel of genomic regions by next-generation sequencing (e.g., using the AVENIO ctDNA analysis kit, Surveillance panel, Roche Sequencing Solutions, Inc., Pleasanton, Calif.). A mutation metric such as AF or MMPM can be determined from the sequencing data. An exemplary data on FIGS. 1, 2 and 3 demonstrates that the number of variants and MaxAF detected in patient blood samples prior to initiation of treatment can predict the benefit of treatment with immune checkpoint inhibitor (e.g., nivolumab (BMS-936558), pembrolizumab (MK-3475), cemiplimab (REGN-2810) atezolizumab (MPDL-3280A), avelumab (MSB-0010718C) or durvalumab (MED-I4736), or an antibody against CTLA-4, i.e., ipilimimab (BMS-734016) and tremelimumab (in development). The data also shown a convenient option of using population-based thresholds, i.e., quantiles such as upper quartile (75%) or median (50%) as determinants whether the patient falls into the high mutation burden or low mutation burden category.

One aspect of the invention includes a system for determining the likelihood that a tumor patient will respond positively to treatment with immune checkpoint inhibitor. The system comprises a processor and a non-transitory computer readable medium coupled to the processor, the medium comprising code executable by the processor for performing a method comprising the steps of analyzing sequencing data on biomarkers from Table 1, performing sequence comparison and mutation detection, error correction, determining one or more of the mutation metrics selected from mutation burden, total number of mutations and maximum allele frequency (MaxAF), wherein the mutation burden is determined as a ratio of the number of mutations identified and the number of bases of nucleic acid sequenced, and the maximum allele frequency (MaxAF) is determined as the highest allele frequency of a single mutation among all mutations detected within a single sample and determining whether the mutation metric in the sample falls above or below a predetermined threshold, e.g., a population-based threshold. In some embodiments, if the mutation metric is at or above the threshold, the system classifies the patient as having high mutation metric and optionally outputs a prognosis or therapy recommendation for high mutation metric. At the same time, if the mutation metric is below the threshold, the system classifies the patient as having low mutation metric and optionally outputs a prognosis or therapy recommendation for low mutation metric.

In some embodiments, the computer readable medium, which may include one or more storages devices, comprises a database including a listing of available therapies depending on mutation metric in the patient. The computer readable medium further comprises a program code having instructions to generate a report listing suitable therapies.

The system may comprise various functional aspects such a server including a processor for processing digital data, a memory coupled to the processor for storing digital data, an input digitizer coupled to the processor for inputting digital data, program code stored in the memory and accessible by the processor, a display device coupled to the processor and memory for displaying information derived from digital data, data networking, and one or more informational databases. The databases may include patient data, patient sample data, clinical data including prior treatment data, a list of therapies and therapeutic agents, patient tracking data and the like.

### EXAMPLES

### Example 1.

### Using Mutation Metrics to Analyze Likelihood of Response in Cancer Patients

In a Phase 3, open-label, multicenter randomized controlled trial, NSCLC patients received either Atezolizumab or docetaxel in a second line setting of advanced NSCLC. Blood plasma samples were obtained from 102 patients prior to start of second line therapy. The AVENIO® ctDNA kit, Surveillance Panel (Roche Sequencing Solutions, Pleasanton, Calif.) was used to assess the number of mutations in patient samples. Cell-free DNA (cfDNA) from the patients was isolated and analyzed according to the manufacturer's instructions of the AVENIO® ctDNA Kit. The metrics assessed included the numbers of variants and maximum allele frequency (MaxAF, the highest allele frequency of a single mutation among all mutations detected within a single sample).

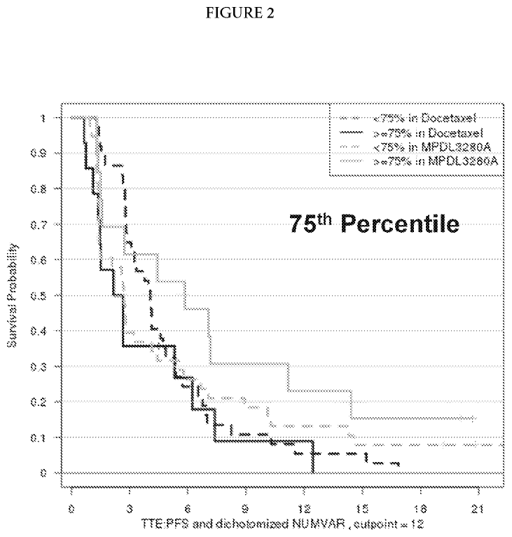

The “number of mutations” metric was assessed on the population level and patients were assessed for progression-free survival (PFS) and overall survival (OS). (FIGS. 1 and 2, Tables 2 and 3) Using median or 75^{th} percentile cut-off, patients treated with Atezolizumab and with number of mutations greater or equal median or 75^{th} percentile have longer progression survival compared to that of chemotherapy, with a HR of 0.67 or 0.49, respectively. Meanwhile, Atezolizumab didn't show more benefit vs chemotherapy in patients with number of mutations fewer than the median or 75^{th} percentile. The HR is 1.12 and 1.05 for <median or <75 percentile, respectively.

TABLE 2

| | | Docetaxel | Atezolizumab | Median | | |
|---|---|---|---|---|---|---|
| | Cutoff value | Arm | Arm | Progression | | |
| | (number of | Events/Total | Events/Total | Free Survival | Hazard | |
| Cutoff | detected | number of | number of | Docetaxel/ | Ratio | |
| point | variants) | patients | patients | Atezolizumab | (HR) | 95% CI |
| <Median | <6.5 | 27/27 | 23/24 | 3.98/2.74 | 1.12 | 0.64-1.96 |
| ≥Median | ≥6.5 | 23/24 | 23/27 | 2.94/2.79 | 0.67 | 0.37-1.21 |

TABLE 3

| | Cutoff | Docetaxel | Atezolizumab | Median | | |
|---|---|---|---|---|---|---|
| | value | Arm | Arm | Progression | | |
| | (number of | Events/Total | Events/Total | Free Survival | Hazard | |
| Cutoff | detected | number of | number of | Docetaxel/ | Ratio | 95% |
| point | variants) | patients | patients | Atezolizumab | (HR) | CI |
| <75^{th} | <12 | 37/37 | 35/38 | 4.07/2.71 | 1.05 | 0.66-1.68 |
| percentile | | | | | | |
| ≥75^{th} | ≥12 | 13/14 | 11/13 | 2.41/5.85 | 0.49 | 0.21-1.14 |
| percentile | | | | | | |

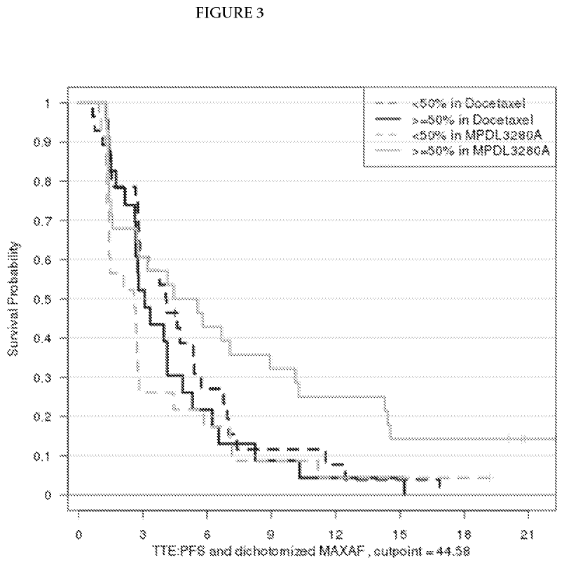

The MaxAF detected in the blood samples was also used to assess its relationship with progression free survival and overall survival. Using median cut-off, patients with MaxAF greater than the median have longer progression free survival when treated with Atezolizumab compared to chemotherapy, with a HR of 0.56 (FIG. 3, Table 4). In patients with MaxAF lesser the median, no benefit was observed on treatment with Atezolizumab vs chemotherapy (HR of 1.45).

TABLE 4

| | | Docetaxel | Atezolizumab | Median | | |
|---|---|---|---|---|---|---|
| | Cutoff value | Arm | Arm | Progression | | |
| | Max Allele | Events/Total | Events/Total | Free Survival | Hazard | |
| Cutoff | Fraction | number of | number of | Docetaxel/ | Ratio | |
| point | (MaxAF) | patients | patients | Atezolizumab | (HR) | 95% CI |
| <Median | <44.58 | 27/28 | 22/23 | 4.09/2.6 | 1.45 | 0.82-2.56 |
| ≥Median | ≥44.58 | 23/23 | 24/28 | 3.09/4.99 | 0.56 | 0.31-1.02 |

### Example 2.

### Using Continuing Monitoring of Mutation Metrics to Analyze Likelihood of Response in Cancer Patients

In this example, the patients' samples from the same Phase 3 trial as described in Example 1 were collected processed an analyzed essentially as described in Example 1. However, the samples were collected serially during the administration of therapy.

Multiple metrics of circulating tumor DNA (ctDNA) were assessed for association with OS. The study has previously demonstrated clinically significant OS benefit in ≥2L NSCLC patients treated with atezolizumab vs docetaxel.

Plasma from 94 patients taken at baseline and at subsequent cycles of therapy every three weeks (C2D1, C3D1, and C4D1) were analyzed retrospectively for ctDNA with the AVENIO ctDNA Surveillance Kit (Roche Sequencing Solutions, Pleasanton, Calif.) Mutations detected in matched PBMC DNA were excluded in correlation analyses with clinical outcomes. ctDNA was measured by allele frequency (AF) or mutant molecules per milliliter (MMPM), and summarized across multiple mutations within a sample by median, mean or maximum. Concordance between these per-sample metrics and PFS/OS were assessed using C index, which is equivalent to AUC under a Receiver Operating Characteristic (ROC) curve.

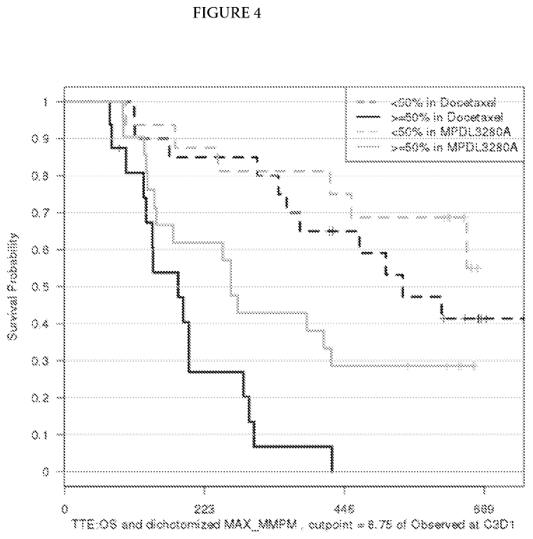

Using absolute and relative change from baseline of these metrics showed various levels of association with OS for both treatment arms as shown in FIG. 4. In FIG. 4 shows overall survival (OS) of patients based on a mutation characteristic measured during treatment. Specifically, samples were collected and assessed at drug administration cycle 1, 2, or 3 or 4 taken in each case, on day 1 (C1D1, C2D1, C3D1, and C4D1). As noted, maximum MMPM (maxMMPM) at C3D1 (sample taken on day 1 or the 3^{rd} round of administration corresponding to 6 weeks of therapy) had comparable association with OS in both treatment arms (atezolizumab arm C index=0.74 and docetaxel arm C index=0.73). Using the max MMPM at C3D1, the patients were dichotomized at a median for the entire cohort of 8.75 maximal MMPM. Survival data (OS) showed that the 1-year survival rate in the atezolizumab arm among MMPM low vs MMPM high patients was 0.81 vs 0.43. In the docetaxel arm, the 1-year survival rate among MMPM low vs MMPM high patients was 0.7 vs 0.07. Because mutation metrics obtained from overall ctDNA levels were associated with OS in metastatic NSCLC, ctDNA is a promising non-invasive blood based biomarker in the metastatic setting of NSCLC.

While the invention has been described in detail with reference to specific examples, it will be apparent to one skilled in the art that various modifications can be made within the scope of this invention. Thus the scope of the invention should not be limited by the examples described herein, but by the claims presented below.

1. A method of treatment of a cancer patient comprising the steps of:
(a) isolating nucleic acids from a cell-free blood sample obtained from the patient;
(b) in the isolated nucleic acid, determining the sequence of at least a portion of each of the biomarkers listed in Table 1;
(c) comparing the sequence determined in step (b) to the reference sequence and identifying mutations;
(d) determining a mutation metric from the mutations identified in step (c);
(e) administering an immunotherapy agent if the mutation metric is high and not administering the immunotherapy agent if the mutation metric is low.

2. The method of claim 1, wherein the sequence of at least a portion of each of the biomarkers listed in Table 1 is determined by a method comprising:
(a) attaching adaptors comprising barcodes to the isolated nucleic acid to generate adapted nucleic acid;
(b) amplifying the adapted nucleic acid to generate amplified non-uniquely tagged progeny polynucleotides;
(c) contacting the amplified nucleic acid with capture probes to capture the amplified nucleic acid comprising at least a portion of each of the biomarkers listed in Table 1;
(d) sequencing the nucleic acid captured in step (c).

3. The method of claim 1, wherein the mutation metric is selected from mutation burden, allele frequency (AF), maximum allele frequency (MaxAF), number of mutant molecules per milliliter (MMPM) and maximum MMPM (MaxMMPM).

4. The method of claim 3, wherein the mutation metric is mutation burden determined as a ratio of the number of mutations identified to the number of bases of nucleic acid sequenced.

5. The method of claim 3, wherein the mutation metric is maximum allele frequency (MaxAF) determined as the highest allele frequency of a single mutation among all mutations detected within a single sample.

6. The method of claim 3, wherein the mutation metric is the number of mutant molecules per milliliter (MMPM) determined as MMPM=AF×HG/V where AF is allele frequency of a particular allele; HG is input haploid human genome equivalent calculated as (extracted mass of DNA in nanograms)×(300 human genome equivalents/nanogram); and V is the volume of plasma in milliliters.

7. The method of claim 3, wherein the mutation metric is the maximum number of mutant molecules per milliliter (MaxMMPM) determined as MaxMMPM=maxAF×HG/V where maxAF is maximum allele frequency among the alleles in the sample; HG is input haploid human genome equivalent calculated as (extracted mass of DNA in nanograms)×(300 human genome equivalents/nanogram); and V is the volume of plasma in milliliters.

8. The method of claim 1, wherein the patient is diagnosed with one of carcinoma, sarcoma, myeloma, leukemia or lymphoma

9. The method of claim 1, wherein the immunotherapy agent is an immunomodulating antibody selected from a group consisting of anti-PD-1, anti-PD-L1 and anti-CTLA-4.

10. (canceled)

11. A method of determining whether a cancer patient is likely to have a benefit from a therapy with an immune checkpoint inhibitor, the method comprising the steps of:
(a) isolating nucleic acids from a cell-free blood sample obtained from the patient;
(b) in the isolated nucleic acid, determining the sequence of at least a portion of each of the biomarkers listed in Table 1;
(c) comparing the sequence determined in step (b) to the reference sequence and identifying mutations;
(d) determining a mutation metric from the mutations identified in step (c);
(e) determining that the patient is likely to have a benefit from a therapy with an immune checkpoint inhibitor if the mutation metric is high and determining that the patient is not likely to have a positive response to a therapy with an immune checkpoint inhibitor if the mutation metric is low.

12. The method of claim 11, wherein the sequence of at least a portion of each of the biomarkers listed in Table 1 is determined by a method comprising:
(a) attaching adaptors comprising barcodes to the isolated nucleic acid to generate adapted nucleic acid;
(c) amplifying the adapted nucleic acid to generate amplified non-uniquely tagged progeny polynucleotides;
(d) contacting the amplified nucleic acid with capture probes specific for least a portion of each of the biomarkers listed in Table 1 to capture the amplified nucleic acid;
(d) sequencing the captured nucleic acid.

13. The method of claim 11, wherein the mutation metric is selected from mutation burden, allele frequency (AF), maximum allele frequency (MaxAF), number of mutant molecules per milliliter (MMPM) and maximum MMPM (MaxMMPM).

14. The method of claim 11, wherein the mutation metric is Max MMPM.

15. The method of claim 11, wherein the mutation metric is low if it falls below the median of the mutation metric of patients with the same tumor type.

16. The method of claim 11, wherein the cell-free blood sample is obtained from the patient during the therapy with an immune checkpoint inhibitor.

17. The method of claim 11, further comprising a step of administering the checkpoint inhibitor to the patient if it has been determined that the patient will benefit from the therapy with the inhibitor.

18. The method of claim 11, further comprising a step of ceasing administration of the checkpoint inhibitor to the patient if it has been determined that the patient will not benefit from the therapy with the inhibitor.

19. The method of claim 11, further comprising a step of administering an alternative therapy to the checkpoint inhibitor to the patient if it has been determined that the patient will not benefit from the therapy with the inhibitor.

20. The method of claim 1, wherein the mutation metric is high if it falls on or above the median of the mutation metric of patients with the same cancer type.

21. The method of claim 1, wherein the mutation metric is high if it falls on or above the top quartile of the mutation metric of patients with the same cancer type.

22. The method of claim 9, wherein the anti-PD-1 antibody is nivolumab administered every 4 weeks at 480 mg or every 2 weeks at 240 mg.

23. The method of claim 9, wherein the anti-CTLA4 antibody is ipilimumab administered at 10 mg/kg every 3 weeks.

24. The method of claim 9, wherein the anti-PD-L1 antibody is atezolizumab administered at 1200 mg every 3 weeks.

25. The method of claim 19, wherein the alternative therapy is chemotherapy with a cytotoxic compound
